(19) United States
(12) Patent Application Publication
(10) Pub. No.: US 2012/0322751 A1
(43) Pub. Date: Dec. 20, 2012

(54) USE OF RHAMNOLIPIDS AS A DRUG OF CHOICE IN THE CASE OF NUCLEAR DISASTERS IN THE TREATMENT OF THE COMBINATION RADIATION INJURIES AND ILLNESSES IN HUMANS AND ANIMALS

(76) Inventor: Goran Piljac, Zagreb (HR)

(21) Appl. No.: 13/582,184

(22) PCT Filed: Feb. 23, 2011
(86) PCT No.: PCT/US11/25843

§ 371 Date: Aug. 31, 2012

(30) Foreign Application Priority Data
Mar. 1, 2010 (HR) P20100108A

(51) Int. Cl. A61K 31/7028; A61P 31/00; A61P 9/10; A61P 25/24; A61P 17/00

(52) U.S. Cl. 514/25

(57) ABSTRACT

This invention is related to the use of one or more rhamnolipids based on structure Formula 1 where the composition of the mixture is useful in the treatment of combination radiation illnesses and regeneration of cells and/or tissues by different application ways of any desirable mentioned rhamnolipids. These rhamnolipid composition mixture are very suitable in the treatment of combination radiation injuries and illnesses and for the regeneration of human and animal cells and tissues after nuclear catastrophes; comprising two or more injuries/illnesses and for cell and tissue regenerations which include radiation damages combined with burns, mechanical injuries, infections of digestive system, infections of lungs, neutropenia, sepsis, atherosclerosis, depression, schizophrenia, atopic eczema and other illnesses in connection with radiation injuries.

### THE SUBJECT OF THE INVENTION

This invention is related to the use of rhamnolipids in the treatment of combination illnesses and injuries after the explosion of atomic bomb, atomic devices or nuclear plants. Combination illnesses or injuries might be: radiation illnesses, burn injuries of the skin and mucous membranes, mechanical injuries, infections of respiratory and digestive system, the injuries of hematological cardiovascular and nervous system.

### Technical Problem

Under the term combination injuries caused by radiation after explosion of atomic bomb, nuclear device(s) or nuclear power plants include: burns, mechanical injuries, depressions, neutropenias septic infections, atherosclerosis, lost of working abilities atopic eczemas and other illnesses caused by nuclear catastrophe. These illnesses and injuries are significantly different with regard to symptoms and treatment in comparison with the treatment of the same illness not caused by explosion of nuclear bomb or nuclear devices. Pernicious combination effect on humans and animals after nuclear disaster is much more disastrous in comparison with any of the mentioned illnesses distinctly and a clinical picture in combination radiation injuries and illnesses is totally different. The effect of drugs and the ways of treatment which are usually effective in particular of the same non-irradiated injuries and illnesses, are not applicable and are totally different. Combination radiation injuries and illnesses caused by nuclear disaster(s) are completely new illness and up today there are no effective drugs for their treatments.

There Exists Necessity for the Drug to Cure all, or at Least Most of the Illnesses and Injuries Caused by Nuclear Catastrophe.

Burn wounds, wounds caused by mechanical injuries, wounds cause by pressure or injuries caused by radiation alone have different pattern and distribution of lethality among animals and humans in comparison with the same illnesses and injuries after combination radiation injuries caused by explosion of atomic bombs, nuclear devices and disasters in nuclear power plant stations.

The rhamnolipids (BAC-3) presented in this invention have special characteristic features, which might be of special value and convenience in the treatment of all or the most of combination illnesses after explosion of atomic bomb or nuclear devices.

### PRESENT STATUS OF TECHNOLOGY

The previous inventions regarding rhamnolipids discovered by Goran Piljac and his collaborators are described in following patents which are relevant for determination of the present status of technology:
U.S. Pat. No. 5,455,232: Goran Piljac, Visnja Piljac; “Pharmaceutical preparation based on rhamnolipids”;
U.S. Pat. No. 5,466,675: Goran Piljac, Visnja Piljac; “Immunological activity of rhamnolipids”;
U.S. Pat. No. 5,514,661: Goran Piljac, Visnja Piljac; Immunological activity of rhamnolipids;
U.S. Pat. No. 7,129,218: Stipcevic Tamara, Piljac Tihana, Piljac Jasenka, Dujmic Tatjana, Piljac Goran; “Use of rhamnolipids in wound healing, treatment and prevention of gum disease and periodontal regeneration”;
U.S. Pat. No. 7,262,171: Tatjana Piljac, Goran Piljac; “Use of rhamnolipids in wound healing, treating burn shock, atherosclerosis, organ transplants, depression, schizophrenia and cosmetics”;
CA 2,195,419: Goran Piljac, Visnja Piljac; “Immunological activity of rhamnolipids”;
CA 2,129,542: Goran Piljac, Visnja Piljac; “Pharmaceutical preparation based on rhamnolipids”;
JP 3860206: Goran Piljac, Visnja Piljac; “Pharmaceutical preparation based on rhamnolipids”;
AU 747088: Tatjana Piljac, Goran Piljac; “Use of rhamnolipids in wound healing, treating burn shock, atherosclerosis, organ transplants, depression, schizophrenia and cosmetics”;
EP0630252: Goran Piljac, Visnja Piljac; “Pharmaceutical preparation based on rhamnolipids against dermatological diseases”; EP0771191: Goran Piljac; Visnja Piljac; “Immunological activity of rhamnolipids”; and international (PCT) applications of patents: PCT/US99/03714, Tatjana Piljac, Goran Piljac “Use of rhamnolipids in wound healing, treating burn shock, atherosclerosis, organ transplants, depression, schizophrenia and cosmetics”; and PCT/US00/17875, Stipcevic Tamara, Piljac Tihana, Piljac Jasenka, Dujmic Tatjana, Piljac Goran; “Use of rhamnolipids in wound healing, treatment and prevention of gum disease and periodontal regeneration”.

However In all previous patents and patent applications, there are no direct or indirect proofs that rhamnolipids might be drug of choice in the treatment of combination illnesses after nuclear catastrophe.

### Today Approach in the Treatment of Irradiated Patients

Today the approach in the treatment of combination injuries is still directed to separate injured organs in the body of injured person using different ways of treatment for the each separate organ. During different scientific meetings it was concluded that it is imperative to find new drug which might be able at the same time to cure all or most illnesses provoked by explosion of atomic bomb, nuclear devices or nuclear plant stations.

Critical obstacle in further development of adequate treatment is the lack of specific drug which might have possibility to ameliorate or cure all symptoms in illnesses and injuries caused by radiation-traumalological-inflammatory processes after exposition to the combination effects after explosion of atomic bombs or nuclear devices.

New drug should have ameliorative or curative effects in combination radiation injuries or illnesses after explosion of atomic bomb, nuclear devices and/or nuclear plant power stations as follows: decrease of, deaths, restoration of normal hematological parameters, normalization of cytokines' arrangement, reduction of bacteria presence, healing of burn wounds, decreasing the wound size, improvement of cognitive tests, curing most of the injured organs, antimicrobial activity, modulation of inflammatory processes by inhibition of inflammatory enzymes, diminution of time treatment by increasing total body proteins in patients, reversing inflammatory processes by invention of the mechanism of combination radiation injuries by immunological processes, using this invention in preventing sepsis, discovering the mechanism of counter-measures in preventing sepsis, speeding up the healing injuries and burn healing caused by nuclear disasters. The drug of choice should also have an acceptable price, be relatively cheap and must be stabile for many years. Except described characteristics of rhamnolipids (BAC-3) in the treatment of different illnesses it is proved that other characteristics of rhamnolipids (BAC-3) confirm that rhamnolipids (BAC-3) are the compounds with potential application in the treatment of combination radiation injuries and illnesses provoked by the explosion of atomic bombs or explosion of nuclear devices.

### ESSENCE OF THE INVENTION

This invention is related to the use of rhamnolipids' composition compromising one or more rhamnolipids of the structure of Formula 1,

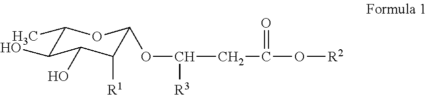

wherein:
R¹═H, unsubstituted α-L-rhamnopyranosyl, α-L-rhamnopyranosyl substituted at the 2 position with
   a group of formula —O—C(═O—CH═CH—R⁵, or —O—C(═O)—CH═CH—R⁵;
R²═H, lower alkyl, —CHR⁴—CH₂—COOH or —CHR⁴—CH₂—COOR⁶;
R³=—(CH₂)ₓ—CH₃, wherein x=4-19;
R⁴=—(CH₂)_{y}—CH₃, wherein y=1-19;
R⁵=—(CH₂)_{z}—CH₃, wherein z=1-12; and
R⁶=lower alkyl,

and where said composition is used for the treatment of humans and/or animals injured after nuclear catastrophe by different modes of application: intravenously, subcutaneously, intramuscular, per os,

This said composition is given in effective dose to the people or animals who need this treatment. This composition is extremely convenient in the treatment of combination illnesses caused by intentional or non-intentional nuclear catastrophe following the explosion of atomic bomb, nuclear devices or nuclear power plant station comprising two or more injuries or illnesses; outstandingly the combination of radiation illness with burn injuries, mechanical injuries, infection of digestive system, infection of respiratory system neutropenia, sepsis, atherosclerosis, depression, atopic eczema and other illnesses linked to radiation.

### DETAILED DESCRIPTION OF INVENTION

The number of victims after nuclear attack estimates that only 15-20% injured will be exposed only to the radiation. At the same time 65-70% victims exposed to radiation will suffer from traumatic injuries. These estimations are based on data achieved after atomic attacks on Hiroshima and Nagasaki when 60-70% irradiated victims were mechanically injured. In the case of Chemobyl disaster in 1986, 10% of 237 victims suffered from burns and received significant doses of radiation.

Although the researches are limited, several studies showed that traumatic injuries increase rate of growth of deaths caused by acute radiation syndrome. In the early research on dog model, the radiation was combined with burn injuries. The burn injuries alone were not lethal. Radiation with (1 Gy) caused only 12% rate of lethality. Together, these two different injuries caused 75% rate of lethality. The similar research was performed on rats having burns of 31-35% surface of total skin area. These burns alone caused lethality in 50% rats and the animals received sub-lethal doses of irradiation (1 Gy and 2.5 Gy). In these rats whose skin surface was burned (31-35%) and irradiated with 1 Gy, the lethality was increased to 65% and if irradiated with 2, 5 Gy the lethality was nearly 100%. When burns in rats were not lethal 16-20% of the total surface skin area, and were irradiated with 5.0 Gy (LD₂₀), 75% animals died from combination effects.

Albeit the mechanisms responsible for synergism between traumatic injuries and irradiation are not yet known, it is obvious that one of the very pronounced reasons is greater submissiveness to infections. Traumatic injury alone decreases resistance to infections, and this effect is additionally aggravated by irradiation. In combination of sub lethal doses of irradiation, the sub lethal burns or wounds synergistically causing bacteria relocation of digestive system into the blood and in this way increase the rate of lethality. The death caused by bacterial infections usually emerges 2-4 days after combination radiation injuries and is dependent on radiation doses. Also open wounds enhance the possibility of the infection development.

In the combination with irradiation the morbidity and mortality caused by the attacks of infective initiators is essentially aggravated. The infections may be provoked by infective initiators naturally occurring in environment or could be introduced as biological weapons. Because irradiation debilitate immunological response, and compromise natural barriers against infection (e.g. the layers of epithelial cells in digestive system and lungs), the people and animals are sensitive to the pathogenic microorganisms. When radiation is combined with exposition to the very infective pathogenic microorganisms, the reduced number of microorganisms is sufficient to produce infection and clinical manifestations are much more serious, as it is confirmed in animal models.

Although the research of combination radiation injuries up to now was relatively limited, the existing data shows that combination radiation injuries are more lethal in comparison of particular injuries alone. Traumatic injuries in combination with irradiation increase the rate of lethality and the healing is more difficult. The injuries caused by radiation increase tendency for infections, nuclear attack during endemic diseases will significantly increase lethality. Synchronously or subsequently exposition to biological weapons will be destructive. Chemicals, whether intentionally or non-intentionally released also could aggravate biomedical effects of radiation. Medical effects of this therapeutic approach for the treatment of combination injuries must be explored to the bottom.

Previous experience showed that nuclear explosions caused not only exposition to radiation but also different injuries such as wounds and burns. The collapse of medical infrastructure after nuclear attack may increase risk of contagious diseases. According to well informed sources the cause of infectious diseases may be endemic opportunistic pathogens or intentionally introduced biological weapons. Chemicals from local industry or storages or from chemical weapons may additionally complicate injuries produce by nuclear attack. Traumatic injuries, pathogens and chemicals will additionally aggravate biomedical consequences of irradiation. To be fully prepared for nuclear or radiological attack it is necessary to understand potential interactions, medical consequences and therapeutic options for the treatment of injuries and illnesses in the case of combination effects of nuclear catastrophes.

However to this day there is no proof if rhamnolipids (BAC-3) have simultaneous beneficial effects in combination illnesses and injuries caused by explosion of nuclear bombs, nuclear devices and/or nuclear plant power stations and which are, according to experts' opinion, new illnesses. Therefore the aim of this research was to determine if rhamnolipids (BAC-3) have abilities which directly or indirectly show that these compounds are the drugs of choice in the case of intentional or non-intentional nuclear catastrophes.

This basic aspect is related to the use of rhamnolipids' composition compromising one or more rhamnolipids of the structure Formula 1,

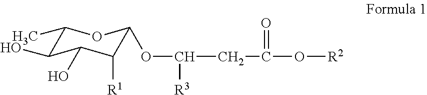

wherein:

R¹═H, unsubstituted α-L-rhamnopyranosyl, α-L-rhamnopyranosyl substituted at the 2 position with
a group of formula —O—C(═O—CH═CH—R⁵, or —O—C(═O)—CH═CH—R⁵;
R²═H, lower alkyl, —CHR⁴—CH₂—COOH or —CHR⁴—CH₂—COOR⁶;
R³=—(CH₂)ₓ—CH₃, wherein x=4-19;
R⁴=—(CH₂)_{y}—CH₃, wherein y=1-19;
R⁵=—(CH₂)_{z}—CH₃, wherein z=1-12; and
R⁶=lower alkyl,
   and where said composition is used for the treatment of humans and/or animals injured after nuclear catastrophe by different modes of application: intravenously: in text (i/v), subcutaneously: in text (s/c), intramuscular: in text (i/m), per os: in text (p/o) and that humans and animals who need such treatment receive effective doses of said composition. Such composition is extremely convenient in the treatment of combination radiation injuries following intentional or non-intentional nuclear catastrophe in humans and animals because of the explosion of atomic bomb, atomic devices or nuclear power plant station comprising two or more injuries or illnesses; outstandingly the combination of radiation illness with burn injuries, mechanical injuries, infection of digestive system, infection of respiratory system neutropenia, sepsis, atherosclerosis, depression, atopic eczema and other illnesses linked with radiation.

The desirable choice for preparing composition for the treatment is the use of rhamnolipids of Formula 1 where said rhamnolipid is:

α-L-rhamnopyranosyl-(1,2)-α-L-ramnopyranosyl)-3-hidroksidecanoyl-3-hidroksidecanoic acid and has the following structure Formula 2:

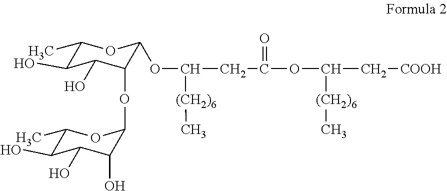

For the treatment the desirable use of composition of Formula 1 and Formula 2 are those, wherein said one or more rhamnolipids of Formula 1 are selected from the group consisting of compounds of Formula 1 wherein:
R¹=—O—C(═O)—CH═CH—R⁵, R²=—CHR⁴—CH₂—COOH, R³=—(CH₂)₆—CH₃, R⁴=—(CH₂)₂—CH₃, and R⁵=—(CH₂)₆—CH₃, or;

R¹=α-L-rhamnopyranosyl substituted at the 2-position by —O—C(═O)—CH═CH—R⁵, R²=—CHR⁴—CH₂—COOH, R³=—(CH₂)₆—CH₃, R⁴=—(CH₂)₆—CH₃, and R⁵=—(CH₂)₆—CH₃, or;

R¹=—O—C(═O)—CH═CH—R⁵, R²=—CHR⁴—CH₂—COOCH₃, R³=—(CH₂)₆—CH₃, R⁴=—(CH₂)₂—CH₃, and R⁵=—(CH₂)₆—CH₃, or;

R¹=α-L-rhamnopyranosyl substituted at the 2-position by —O—C(═O—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

It is important that chosen rhamnolipid compounds are in the composition of clear liquids, physiological solution, suspension, dispersion, emulsion, cream, paste, powders, infusion fluids, ointments, tinctures, lotions, capsules, pills, suppositories, depot forms, tablets and gels for the purpose of the mentioned treatments. It is obvious that every kind of treatment must contain enough effective quantity of the any rhamnolipid composition depending of chosen composition and the way of application.

Likewise, the compositions for topical administration embrace one or more rhamnolipids of Formula 1 and suitable carrier of the composition or any of their combination chosen from: eucerin, olive oil, sunflower oil, and other eatable oils, polyethylene glycol, alcohol, petrolatum, water, standard pharmaceutical alcoholic mixtures, physiologic solutions, lanoline, glycol stearate, milk and other ointments, propylene glycol, glycerol, stearic acid, vitamins A, D, E, and K in combination with oils and other standard pharmaceutical compositions used for topical applications.

It is recommended that above mentioned carriers comprise from 0.001 to 5% by weight of said one or more rhamnolipids of the Formula 1 based on total weight of the composition. It is preferred that above mentioned carriers comprise from 0.01 to 10% of said one or more rhamnolipids of the Formula 1 based on total weight of the composition.

The way of treatment of said composition should be following administration left enough time for the effective achievement of the therapy of illnesses which are the consequence of combination radiation effect. Therefore it will be necessary after nuclear catastrophe to administer topically one or more rhamnolipids in described way.

Except topical applications of said rhamnolipids, any of the chosen rhamnolipids of Formula 1 could be administered parenterally alone or in the form of the combination with one or more other said rhamnolipids. Parenterally administration of anyone of the said rhamnolipids might be i/v, s/c, i/m, and p/o. According to this anyone of the said rhamnolipids of Formula 1 could be administered in the amount from 4 mg to 40 mg per kilogram or preferably 1 mg to 20 mg per kilogram 2-4 times per day of body weight what depends of the route of administration. It is preferably that anyone of the said rhamnolipids of Formula 1 must be administrated in the effective doses enough times in the described compositions.

In these aspects, this invention is also related to the use of said rhamnolipid composition which comprise one or more rhamnolipids of the structure Formula 1:

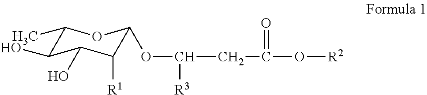

wherein:
R¹═H, unsubstituted α-L-rhamnopyranosyl, α-L-rhamnopyranosyl substituted at the 2 position with
   a group of formula —O—C(═O—CH═CH—R⁵, or —O—C(═O)—CH═CH—R⁵;
R²═H, lower alkyl, —CHR⁴—CH₂—COOH or —CHR⁴—CH₂—COOR⁶;
R³=—(CH₂)ₓ—CH₃, wherein x=4-19;
R⁴=—(CH₂)_{y}—CH₃, wherein y=1-19;
R⁵=—(CH₂)_{z}—CH₃, wherein z=1-12; and
R⁶=lower alkyl,

And wherein said composition is used for the treatment of humans and/or animals suffering from combination radiation injuries which comprise:
Radiation, burns, mechanical injuries and infections of skin, mucous membranes and body organs; and
One or more illnesses or disfunctions chosen from: atherosclerosis, depression, atopic eczema and/or other illnesses caused by irradiation; in a way that humans or animals which needs this kind of treatment receive effective dose of said composition

Desired choice for preparation said compositions for the treatment of above mentioned combination radiation illness is the use of the rhamnolipid of the Formula 1 in a way that said compositions comprise: α-L-rhamnopyranosyl-(1,2)-α-L-ramnopyranosyl)-3-hidroksidecanoyl-3-hidroksidecanoic acid and has the following structure Formula 2:

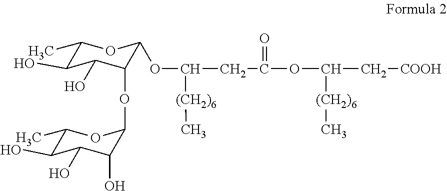

For the treatment the desirable use of composition of Formula 1 and Formula 2 are those, wherein said one or more rhamnolipids of Formula 1 are selected from the group consisting of compounds of Formula 1 wherein:
R¹=—O—C(═O)—CH═CH—R⁵, R²=—CHR⁴—CH₂—COOH, R³=—(CH₂)₆—CH₃, R⁴=—(CH₂)₂—CH₃, and R⁵=—(CH₂)₆—CH₃, or;

R¹=α-L-rhamnopyranosyl substituted at the 2-position by —O—C(═O)—CH═CH—R⁵, R²=—CHR⁴—CH₂—COOH, R³=—(CH₂)₆—CH₃, R⁴=—(CH₂)₆—CH₃, and R⁵=—(CH₂)₆—CH₃, or;

R¹=—O—C(═O)—CH═CH—R⁵, R²=—CHR⁴—CH₂—COOCH₃, R³=—(CH₂)₆—CH₃, R⁴=—(CH₂)₂—CH₃, and R⁵=—(CH₂)₆—CH₃, or;

R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

In further aspects, this invention is also related to the use of said rhamnolipid composition which comprise one or more rhamnolipids of the Formula 1:

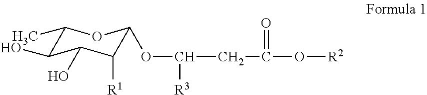

wherein:
R¹═H, unsubstituted α-L-rhamnopyranosyl, α-L-rhamnopyranosyl substituted at the 2 position with
   a group of formula —O—C(═O)—CH═CH—R⁵, or —O—C(═O)—CH═CH—R⁵;
R²═H, lower alkyl, —CHR⁴—CH₂—COOH or —CHR⁴—CH₂—COOR⁶;
R³=—(CH₂)ₓ—CH₃, wherein x=4-19;
R⁴=—(CH₂)_{y}—CH₃, wherein y=1-19;
R⁵=—(CH₂)_{z}—CH₃, wherein z=1-12; and

R⁶=lower alkyl,

wherein:

said compositions are used in humans and/or animals in the procedure of regeneration of cells and tissues: hematopoietic system, epithelia of the skin, mucous membranes epithelia of digestive, respiratory; vascular; cerebral nervous system, osteomuscular system after combination radiation illnesses which compromise radiation, burns, mechanical injuries, infection of the skin, mucous membranes and organs, atherosclerosis, atopic eczema and other illnesses with are caused by irradiation in a way that humans or animals in needing received effective doses of said compositions.

The desirable choice for preparing treatment of regeneration of the cells and tissues composition is the use of the Formula 1 where said rhamnolipid is: α-L-rhamnopyranosyl-(1,2)-α-L-ramnopyranosyl)-3-hidroksidecanoyl-3-hidroksidecanoic acid and has the following structure Formula 2:

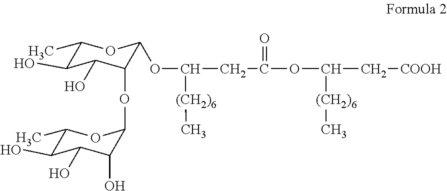

Desirable choice for the forming composition for regeneration of the cells and tissues is the use of rhamnolipid of Formula 1 in the way that it is chosen rhamnolipid:

wherein:
R¹=—O—C(═O)—CH═CH—R⁵, R²=—CHR⁴—CH₂—COOH, R³=—(CH₂)₆—CH₃, R⁴=—(CH₂)₂—CH₃, and R⁵=—(CH₂)₆—CH₃, or;

R¹=α-L-rhamnopyranosyl substituted at the 2-position by —O—C(═O)—CH═CH—R⁵, R²=—CHR⁴—CH₂—COOH, R³=—(CH₂)₆—CH₃, R⁴=—(CH₂)₆—CH₃, and R⁵=—(CH₂)₆—CH₃, or;

R¹=—O—C(═O)—CH═CH—R⁵, R²=—CHR⁴—CH₂—COOCH₃, R³=—(CH₂)₆—CH₃, R⁴=—(CH₂)₂—CH₃, and R⁵=—(CH₂)₆—CH₃, or;

R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

### BRIEF DESCRIPTION OF FIGURES

The understanding of this invention is presented by FIGS. 1-11 which contain:

FIG. 1—UV spectrum of BAC-3;

FIG. 2—Mass spectrum of BAC-3;

FIG. 3—FAB spectrum of BAC-3;

FIG. 4—Structure formula of BAC-3;

FIG. 5—The effect of BAC-3 on MNC proliferation stimulated by 1 μg/ml of PHA;

FIG. 6—The effect of BAC-3 on proliferation of MNC stimulated by 1 μg/ml of SEB;

FIG. 7—The effect of BAC-3 on spleen mouse cells stimulated by 1 μg/ml of lipopolysaccharide;

FIG. 8—BAC-3 significantly reduced the protein synthesis dedicated to collagen synthesis on dose response effect;

FIG. 9—BAC-3 decreases collagen synthesis (2a) but with an increase in overall protein synthesis (2b);

FIG. 10—UV spectrum of BAC-3 after 10 years of production; and

FIG. 11—Mass spectrum of BAC-3 after 10 years of production (a) and other accompanying di-rhamnolipd (b)

### THE EXAMPLES OF PERFORMANCE

### Example 1

### The Production of Rhamnolipid BAC-3 from Strain Ps. Aeruginosa Sp

After fermentation, biomass was separated by Beckman ultracentrifuge at a speed of 60,000 g, and room temperature. After biomass was removed, the original volume, which contained rhamnolipids was filtrated through 0.1 μm filter to remove the rest of bacteria and cells' debris. After that the same volume was ultrafiltrated through 10⁶ molecular filter, following through 10⁵ and 10⁴ molecular filter (Pellicon cassettes) using Millipore system with continuous flow (Millipore, Billerica, Mass., USA). After last filtration all supernatant was collected, re-suspended in the same quantity of Millique filtered water and the procedure was repeated 4 times. In this way the color of the fermented broth was removed and the rest of colorless or slightly colored rhamnolipids were collected in supernatant. Supernatants with rhamnolipids were removed and diluted in distillated water up to the original volume. Prepared solution contained rhamnolipids which were additionally purified with help of adsorption chromatographic system.

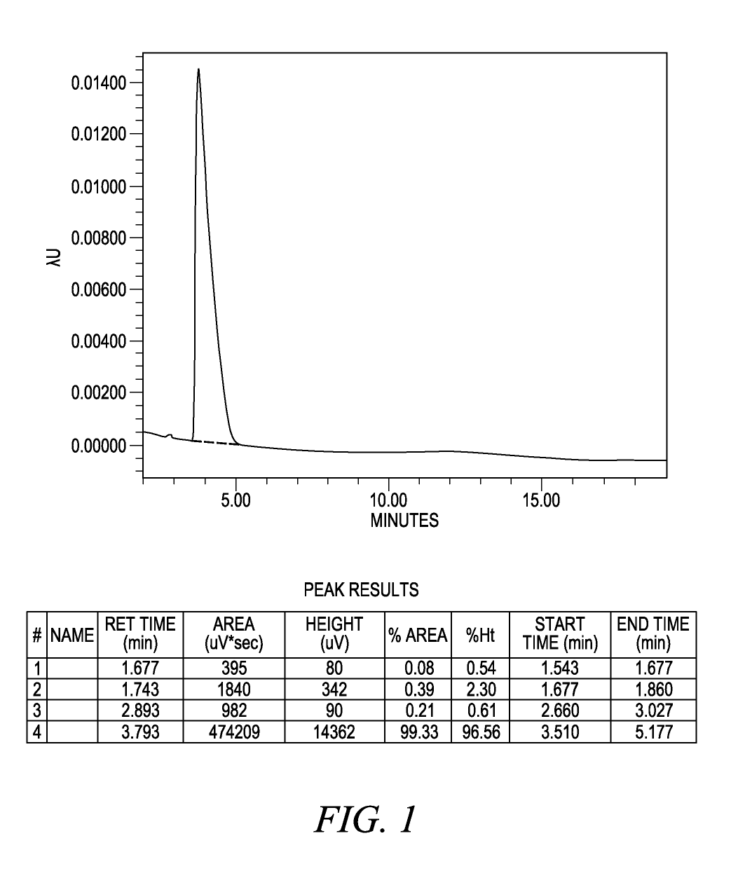

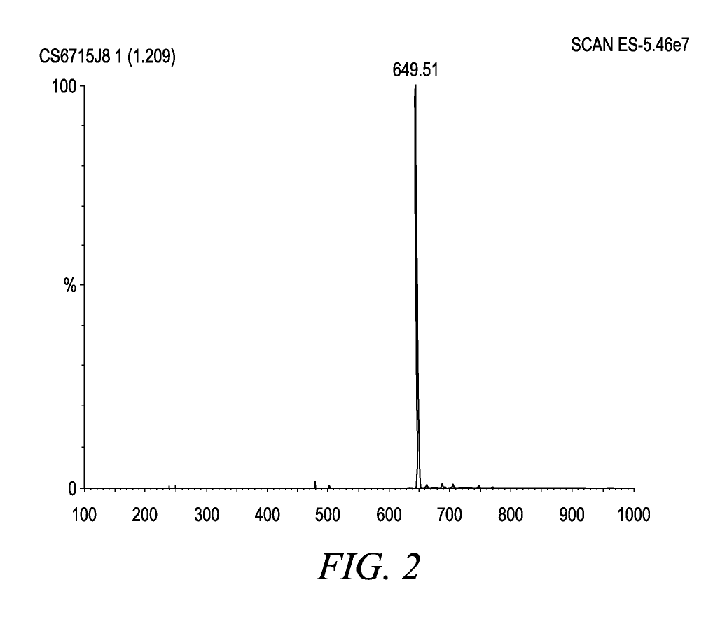

Initial low-pressure liquid chromatography was performed on Pharmacia columns. The columns were filled with adsorption resins Amberlite XAD-7 or XAD-8 (Rohm&Haas, Philadelphia, Pa., USA). The columns with dimension 30×10 cm)Pharmacia) were filled with previously prepared XAD-7 and XAD-8 resins, equilibrated 10 times with the same volume of distilled water The solution which contained rhamnolipids was loaded over resins in columns. The flow rate was 4 column volume per hour. The total adsorption capacity of XAD-7 or XAD-8 resins was 30 liters of dissolved rhamnolipids. After successful adsorption on XAD-7 or XAD-8 resins the column was rinsed with ten times higher volume of distilled water. Rhamnolipids were eluted with gradient 50% of EtOH and 50% H₂O at the same flow rate (4 column volumes per hour) Void volume was removed and rhamnolipids were collected with fraction collector (Pharmacia) in 500 ml bottles. Two bottles contained the highest quantity of di-rhamnolipid (BAC-3) and the other bottles contained the rest of rhamnolipids. All bottles were left over night in refrigerator at +4° C. The rhamnolipids were precipitated and supernatant was removed. The precipitate was freeze dried and was stored for further purification. Preparative and analytical chromatography (HPLC) was performed using reverse phase on Waters HPLC devices The purity of the compound BAC-3 is shown on figure (FIG. 1), and Mass spectrum of BAC-3 on FIG. 2 (FIG. 2).

### Example 2

### FAB Spectrometry

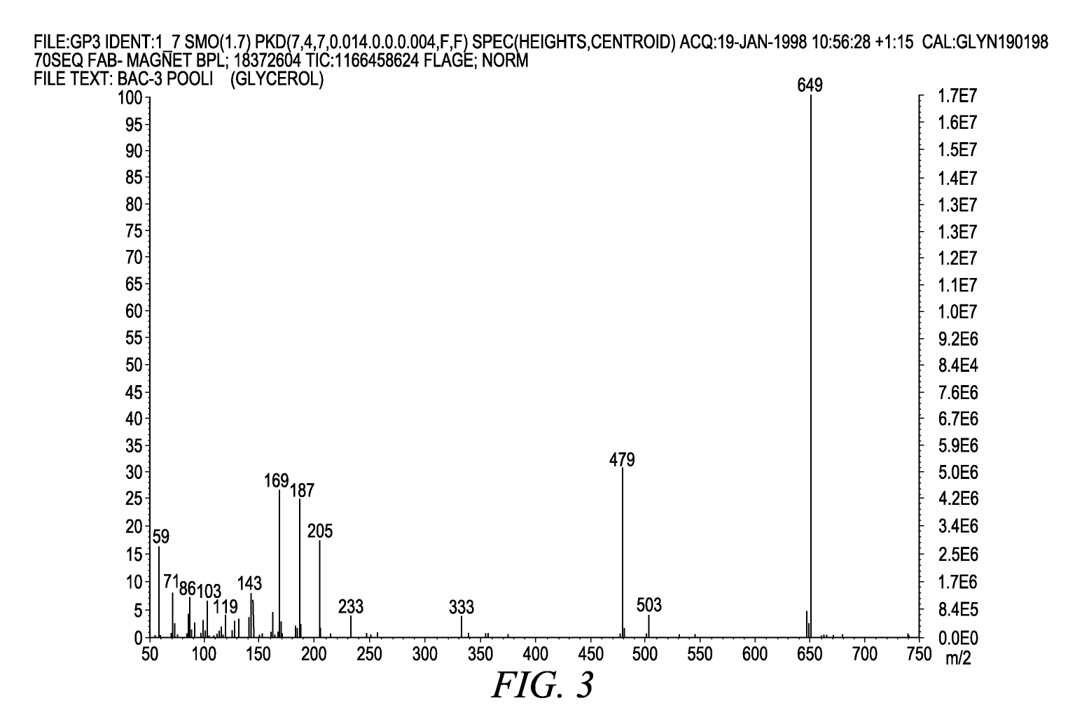

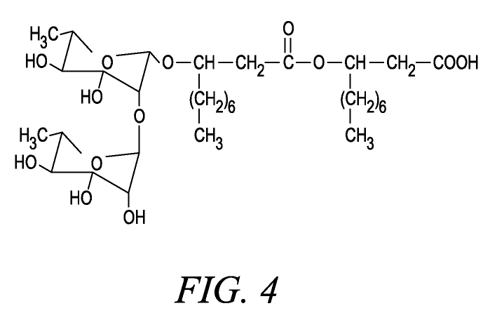

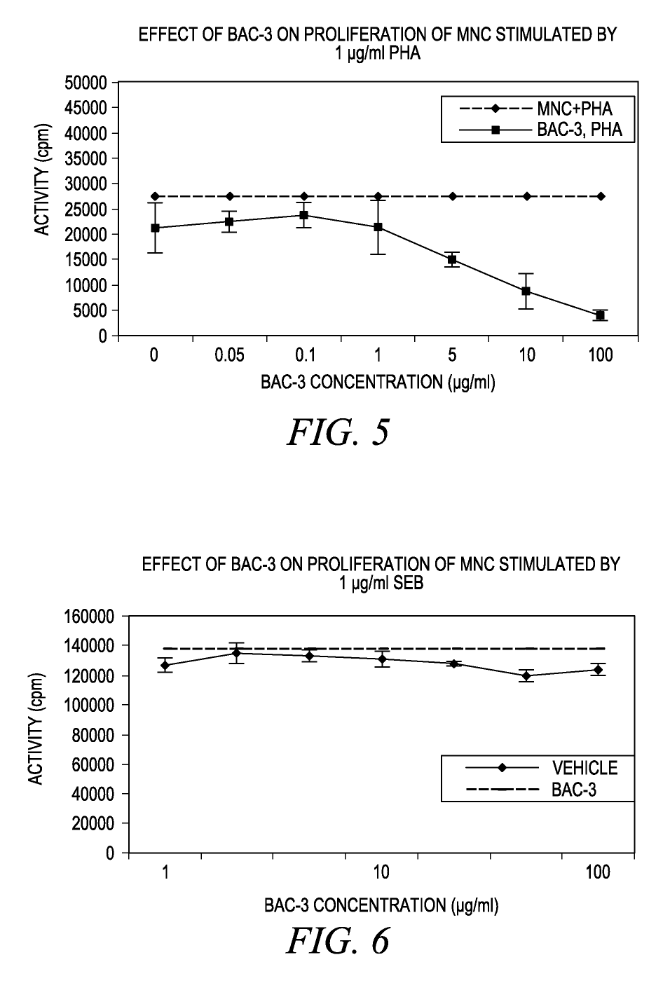

FAB spectrums (Eng: Fast Atomic Bombardment) of BAC-3 were collected by the help of LSIMS/CID methods (Liquid Secondary Ion Mass Spectrometry/Collision Induced Dissociation) on VG70SEQ hybrid mass spectrometer) Micro Mass, Manchester, UK) s EBQQ configuration which was equipped with source of Cesium ions. On figure (FIG. 3) was shown one part of FAB spectrometry and on figure (FIG. 4) was shown structure formula of: α-L-rhamnopyranosyl-(1,2)-α-L-ramnopyranosyl)-3-hidroksidecanoyl-3-hidroksidecanoic acid.

### Example 3

### Leukotriene C4 Synthase

Leukotriene C₄ (LTC₄) synthase is crucial in the formation of LTC₄ from LTA₄. Taken together with other assays for enzymes involved in the lipoxygenase pathway (e.g. 5-lipoxygenase, LTA₄ hydrolase), a locus of action can be established, mechanism of agents' activities which inhibit the formation of the leukotrienes.

Guinea pig lung LTC₄ synthase was used. Test compound and/or vehicle was preincubated with 180 μg/ml enzyme dissolved in phosphate buffer pH 7.8 for 15 minutes at 37° C. The reaction was initiated by addition of 2.5 μg/ml LTA₄ methyl ester for another 30 minute incubation period and terminated by further addition of ice-cold methanol. Determination of the amount of LTC₄ formed was read spectrophotometrically by enzyme immunoassay kit (EIA). Compounds was tested at the concentrations of 1,000; 100; 10; and 1 μM/ml. The used solvent was DMSO. At the highest concentration, the inhibition of leukotriene C₄ synthase with rhamnolipid BAC-3 was 81%.

### Example 4

### The Effect of Rhamnolipfid (BAC-3) on the Proliferation of Human Mononuclear Cells

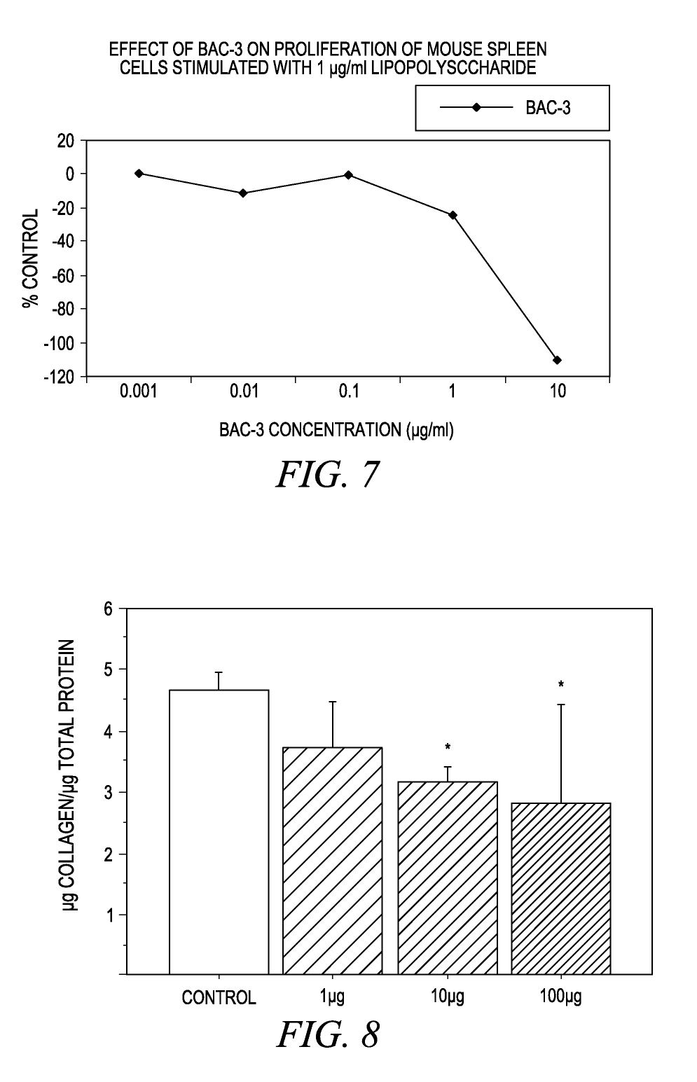

BAC-3 inhibited proliferation of human mononuclear cells (MNC) as a response to different stimulans; normal antigen (tetanus toxin), superantigen Staphylococcal Enterotoxin B and lectin (phytohemaglutinin). Di-rhamnolipid BAC-3 which inhibited the proliferation of MNC as a response to exposition of PHA at 1 μg/ml BAC-3 had IC₅₀=35 μg/ml at the concentration of 5 μg/ml. The inhibition of MNC proliferation is not due to simple toxicity because almost no inhibition is present when high doses of stapyloccocus enterotoxin (SEB) are used as stimulus. (FIG. 6). The effect of BAC-3 on the proliferation of mouse spleen cells is shown on figure (FIG. 7). The molecular target for inhibition is not known because BAC-3 has carbohydrate moiety, and because the inhibition had smallest effect when superantigen was used as a stimulus for T cells.

### Example 5

### Description of the Procedure for Assessment of the Effect of Rhamnolipid BAC-3 on Collagen Synthesis and Total Protein Produced by Human Fibroblasts In Vitro

a) Neonatal human fibroblast culture was established from a sample of neonatal human foreskin obtained by circumcision. After removal of subcutaneous tissue by dissection, the sample was desegregated by trypsinization with 0.25% trypsin and 1 mM EDTA solution (Gibco). Released cells were propagated in 100 mm plastic tissue culture dishes (Corning) in incubator at 37° C. saturated with 5% CO2. The medium, changed twice weekly, was a mixture of 0.2 mg/ml calcium, 4500 mg/l glucose in Dulbecco's modified Eagle's medium (DMEM, Gibco) supplemented with 10% fetal bovine serum FBS (Gemini-Bio-Products), 0.292 mg/ml L-glutamine (Gibco) and antibiotic-antimycotic solution containing 100 U penicillin-G/ml, 100 mcg/ml streptomycin and 0.25 mcg/ml fungizione in normal saline (Gemini-Bio-Products). Primary culture cells were trypsinized. To achieve enough cells of the same passage, the cells were replanted two times during two weeks and transplanted in bigger dishes before incubation started. At density 5×10⁵ NHK 98-009 of fibroblasts of passage 4 was transplanted in Petri dishes in DMEM medium. When cells achieved ca 50% of confluence they were treated with solutions of BAC-3 with 0; 1; 10; 100; 250; and 500 μg/ml and 50 μg/ml of ascorbic acid (Sigma). All solutions were filtrated through 0.2 μm filters (Fisher). The media with or without BAC-3 were changed once after two days. 100 μg of ascorbic acid were added each day in media until achieving final concentration of 50 μg/ml of ascorbic acid in each of Petri dishes. The cells were incubated during period of 4 days at 37° C., in 5% CO₂ incubator.
b) On day 5, medium was removed from dishes and cells were washed two times with 4 ml of 10 mM phosphate buffer PBS (Gibco). The cells attached on the bottom of Petri dishes were scratched out by rubber policeman and were collected in 1 ml PBS (the aliquots of cell matrix)
c) The cells were collected in 1 ml of 10 mM PBS solution and homogenisated with sonicator (Fisher). The whole concentration of proteins were done according Bradford's method and whole concentration of hydroxiproline was determinated according Woessner's method.

### Example 6

### The Effect of Concentration of Rhamnolipid BAC-3 on Collagen Synthesis Expressed as Percent of Total Protein Synthesis

Normal human fibroblasts derived from foreskin were cultured in T-25 flasks and were treated with BAC-3 at concentrations of 1 μg/ml, 10 μg/ml, and 100 μg/ml in the way previously described. Cells were harvested at day 4 and day 7. The medium was decanted and freezed; the cells were scraped from the plate with rubber policeman in normal saline into a small vial and sonicated. One half of the sonicate was hydrolyzed in 6N HCl; hydroxyproline was measured in the hydrolisate to determine collagen synthesis. An aliquot of the remaining sonicate was used for protein determination using Lowry assay. Total collagen per flask was determined multiplying the hydroxiproline value by 7.8. As shown on FIG. 8 rhamnolipid BAC-3 significantly reduced the percent of protein synthesis dedicated to collagen synthesis; there was also a significant dose-response effect.

FIG. 8—Legend: Open bars=control cells; stripped bars=cells treated with rhamnolipid BAC-3. T-bars represent Standard deviation. Asterisks above the bars indicate significant differences from control value. (p<0.05 by ANOVA).

### Example 7

### The Effect of Concentration of Rhamnolipid BAC-3 on Total Collagen Synthesis and Total Protein Synthesis, Expressed as μg of Protein Per Flask

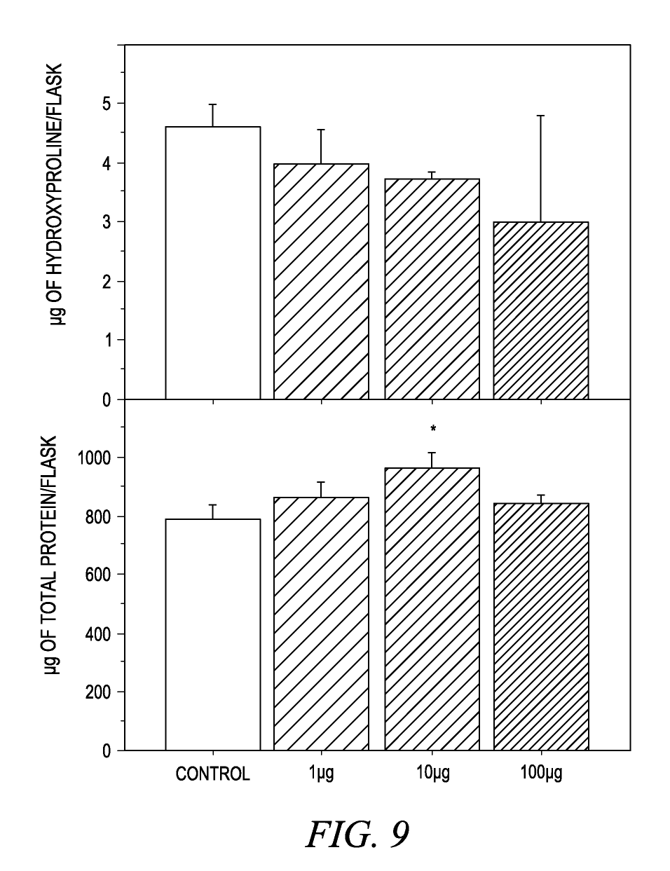

Cells were grown and treated with rhamnolipid BAC-3 as described in example 6. The figure shows that rhamnolipid BAC-3 decreases synthesis of collagen (2a) but within an increase in overall synthesis (2b). (FIG. 9).

Legend: Open bars=control cells; striped bars=cells receiving rhamnolipd BAC-3. T-bars represent Standard deviation. Asteriks above the bars indicate significant differences from control values (p<0.05 by ANOVA).

### Example 8

### Anti-Inflammatory Activities of Rhamnolipid BAC-3 on TPA/pyr Induced Acute Ear Inflammation in Swiss-Webster Mice

The inflammatory responses occur in three distinct phases, each apparently mediated by different mechanisms: an acute one characterized by local vasodilatation and increased capillary permeability, a subacute phase characterized by infiltration of leukocyte and phagocytic cells and a chronic proliferative phase, in which tissue degeneration and fibrosis occur. Acute inflammation generated by TPA (12-o-tetradecanoylforbolacetat/pyridine) induces an acute inflammatory reaction consisting of erythema, edema and polymorphonuclear leukocyte (PMN) infiltration The mechanism of anti-inflammatory mode of action of di-rhamnolipid BAC-3 is unknown. So far, it has been shown that Pseudomonas di-rhamnolipids stimulate, both chemotaxis and chemokinesis of leukocytes). In vitro studies examining the effects of pulmonary surfactant containing hydrophobic apopoproteins on human neutrophils have the important role of the protein component of the surfactant in enhancing the leukocyte chemotaxis, opsonizing bacteria, enhancing macrophage function, and stimulating antibody production. The suggested mode of action predicted that surfactant releases Ca⁺⁺ stores through insertion of channels, depolarization of neutrophils, and activation of G protein-dependent pathway. Correspondingly, it has been recognized that glycolipids (biosurfactants) through their virtue of hidrophilic head bind to proteins, i.e. receptors on cell membranes. Altogether, these results support the hypothesis of the mechanism of action of rhamnolipid BAC-3 consisting of the creation of complexes of BAC-3 with proteins abundantly present in the serum, triggering the cell-signaling and consequently influencing the local environment of the cellular matrix. It has been proposed that rhamnolipid increased the early recruitment of inflammatory cells to the site of inflammation without triggering a persistent, abnormal accumulation of neutrophils. With regards to proliferative phase of inflammation, di-rhamnolipid BAC-3 inhibited proliferation and collagen production of fibroblast, demonstrating antifibrotic activity. It is assumed that further research testing, BAC-3 could prove to be novel agent for suppressing inflammation and find its clinical use in combination radiation injuries and illnesses generated after explosion of atomic bombs or nuclear facilities.

In the experiment it was proven that rhamnolipd (BAC-3) at concentration (1 mg/ml⁻¹, 0.1% w/v) was effective as a topical therapeutic anti-inflammatory agent in the TPA/pyr model of acute dermal ear inflammation in female Swiss-Webster mice over a time-course of 24 hours. The time of maximal reduction of inflammation was dependent on the time of treatment, with respect to induction.

Rhamnolipid (BAC-3) used as pre-treatment on the ear 1 hour before TPA/pyr showed short protective effect by decreasing inflammation for 15% in comparison with control ear. Concurrent treatment with BAC-3 during induction of inflammation with TPA/pyr effectively inhibited inflammation by about 32% at 6 hours in comparison with control ear. Post treatment with BAC-3 two hour after inflammation reversed established inflammation with maximal effect after 11 hours. (32% lower inflammation in comparison with control). The results of this study suggest that topical treatment with single dose of 0.1% solution of BAC-3 in physiological solution one hour before induction of inflammation can prevent creating of swelling for 15%, can lower inflammation for 32% when treatment started at the same time as inflammatory induction and reverse inflammatory process (for 32%) when given 2 hours after inflammation has been induced. It may be possible to increase the anti-inflammatory effect of BAC-3 by use of a different dose formulation, or by repeated treatments. The effect of rhamnolipids (BAC-3) was moderate but sustained.

### Example 9

### The Effect of Rhamnolipid BAC-3 on Delayed Type Hypersensitivity (DTH) in Nude Guinea Pigs

Based on previous results in vitro and in vivo further testing in another species for example, standard guinea pig test system was used prior to any firm conclusions being drawn.

The nude guinea pig is used as test animal because of different animal skins, nude guinea pig skin is claimed to be the one closest resembling human skin. The model was not optimized completely yet, but preliminary data from guinea pigs showed very strong anti inflammatory effect on this model. In DNCB sensitised guinea pigs delayed type hypersensitivity reactions were induced with patches soaked with 0.25% (w/v) DNCB in ethanol/propylene glycol kept on the animals back for 24 hours. The test sites were treated twice daily during 7 days with: Dovonex (commercially available ointment with calcipotriene; 1% (w/w) BAC-3 in US petrolatum. Locobasis (commercially available ointment, identical as carrier for steroids; 1% (w/w) non purified BAC-3 (86%) in US petrolatum; US petrolatum; Preferid (commercially available steroid).

By day 7 the calcipotriene and the 1% BAC-3 test areas were almost healed with only a slight erythema present while the untreated and the vehicle treated areas still had a substantial erythema and induration. Preferid, as well as its control with US petrolatum induced inflammation in the surrounding skin. The test area treated by preferid has been improved by time. The other guinea pig demonstrated similar result with the crude (86%) BAC-3 being intermediary in effect between petrolatum and the pure BAC-3.

### Example 10

### Preservation of the Rhamnolipid (BAC-3) Structure During 10 Years after Production

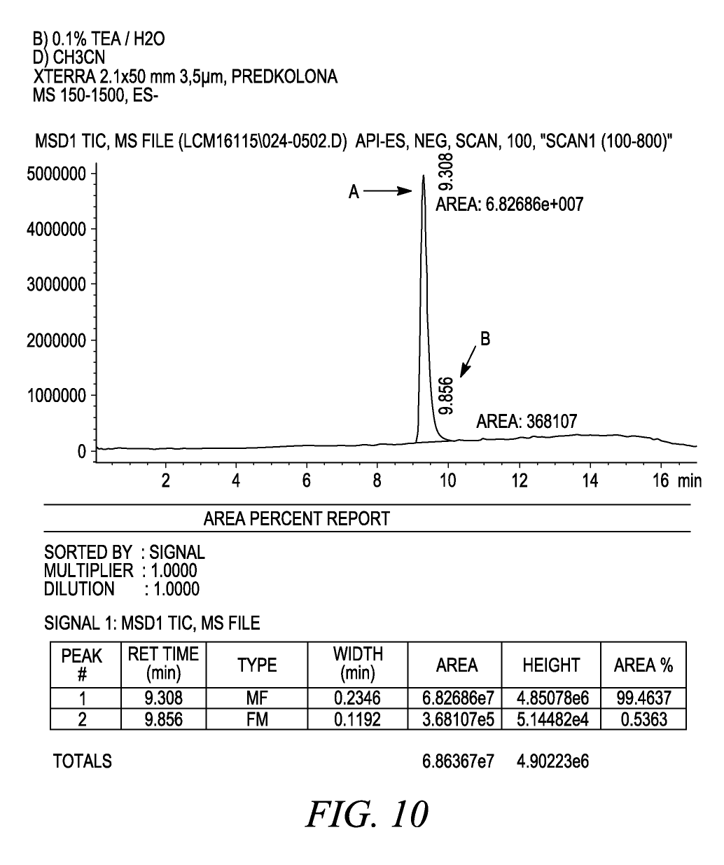

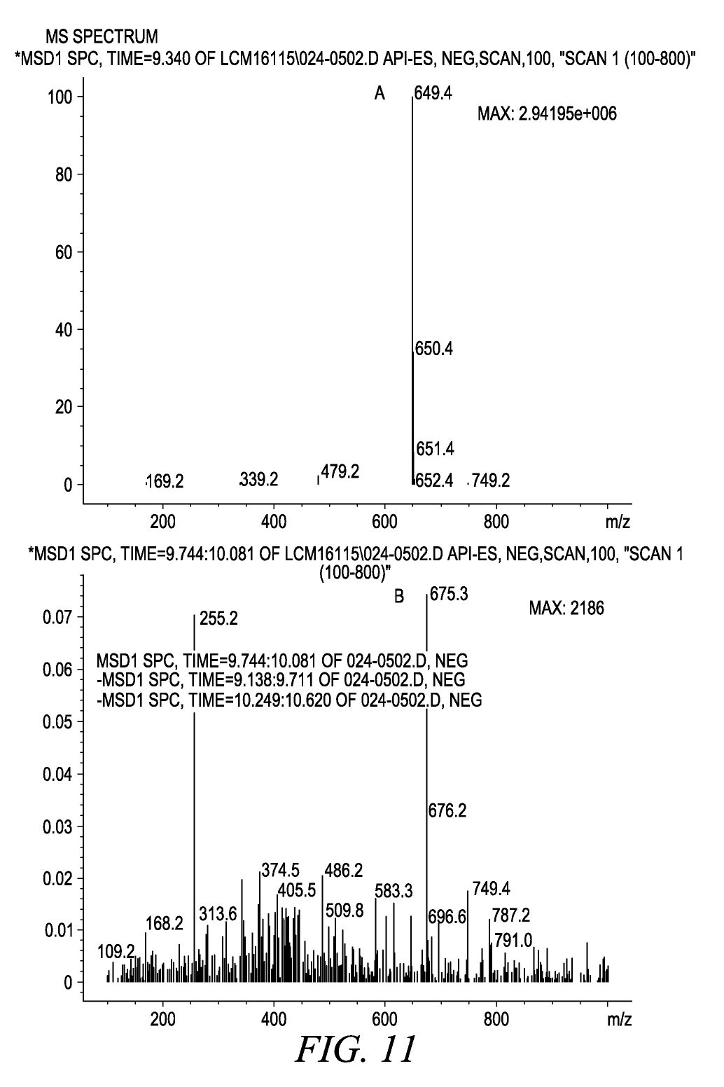

During this research the inventor proofed that rhamnolipid BAC-3, kept under nitrogen N₂ in the glass bottles at room temperature did not change its structure during 10 years. On the figure has been shown the purity of the rhamnolipid BAC-3 (FIG. 10) and mass spectrum (FIG. 11) ten years after production. The production of the rhamnolipid BAC-3 purity>98.5 is very expensive procedure. Therefore one of the most important task of rhamnolipid BAC-3 production is achieving clinically acceptable purity without using HPLC-MS equipment, except for control purposes of the purity and mass spectrum.

### Example 11

### The Rules of the Used Methods During the Treatment of Irradiated Rats

a) Determination of complete blood parameters, cytokines and biochemical data

For experiments is necessary the use of 756 healthy female Harlan Sprague-Dawley rats at the age of three months. From these 756 female rats, 328 rats are necessary for subcutaneous (s/c) application and 328 for per oral (p/o) application of the rhamnolipid BAC-3. It is necessary in all experiments and all animals to determine complete blood parameters. The blood sample is taken from the blood of all 328 animals from the tail vein on the 7th day after the quarantine finished, on the 55 th day, and on the 99 th day, and a day before euthanasia. Blood is taken in order to determine the complete blood count, corresponding biochemical data, and cytokines: CINC-2α/β, CINC-3, GM-CSF, ICAM-1/CD54, IFN-γ, IL-1α/IL-1F1, IL-1β/IL-1F2, IL-2, IL-4, IL-6, IL-10, IL-13, IL-18/IL-1F4, LSelectin/CD62L, TIMP-1, TNFα/TNFSF1A, VEGF. (R&D Systems Inc). Among the listed cytokines are those with pro-inflammatory activity, anti-inflamatory activity, as well as chemokynes, growth factors and adhesion mollecules.
b) The determination of penetration ability of the ointment with rhamnolipid BAC-3.

The penetration ability of the ointment with rhamnolipid BAC-3 is determined on the skin of human donor before any topical application on the places of burn wounds and the wounds on rats' tibia.
c) On all 756 animals it was necessary to perform the following psichological tests: Chronic mild stress; forced swimming test, social interaction test and working memory test. Some animals were used for auditory sensory gatting test.
d) It is necessary to treat all animals with rhamnolipid BAC-3 either s/c or p/o, only 24 hours after irradiation.

### Subcutaneous Application of the Rhamnolipid BAC-3

### Example 12

### First Main Group Consists of 58 Animals Used for Determination of the Parameters Necessary Before Performing Main Experiments

The Animals are Chosen Randomly.

1) Ten animals served as a control group and shall not be treated.
2) Thirty animals are necessary for determination of LD 50/60 parameters according the following doses: a) ten Sprague Dawley female rats in each group underwent irradiation of the whole body doses:|
a) 6.9 Gy
b) 6.1 Gy
c) 7.5 Gy

Previously determined whole body irradiation dose value LD50/30 for Sprague-Dawley female rats was 6.9 Gy.

2) Eighteen animals are not exposed to irradiation, burn wounds, wounding of tibia and surrounding tissue by scalpel and were not infected with C. albicans. This group will serve to determine potential side effects of different concentrations of rhamnolipid BAC-3. In this group animals are treated twice daily: every 12 hours with
a) saline solution (6 animals) as placebo;
b) 1.2 mg/kg of BAC-3 in saline solution;
c) 12 mg/kg of BAC-3 in saline solution.

### Example 13

### The Second Main Group has 270 Female Sprague-Dawley Rats

The animals are chosen randomly and treated twice daily (every 12 hours).

After determination of LD 50/60, in the first group of animals are: a) 90 animals irradiated with LD 50/60; b) 90 animals irradiated with 2 Gy lower irradiation value of LD 50/60; c) 90 animals irradiated with 4 Gy lower irradiation value of LD 50/60.

All 270 animals were divided in 5 sub-groups. Animals from each sub-group shall be treated during 90 days. Every animal is treated twice daily every 12 hours with two different concentration of rhamnolipid BAC-3 and with placebo. At the same time burned animals shall be treated with ointment with low penetration possibility. The concentration of BAC-3 in these ointments is (0.1% w/w) or (1% w/w) or placebo ointment. The tibia wounds are treated with ointment with high penetration possibility of rhamnolipid BAC-3 (0.1% w/w) or (1% w/w) or placebo ointment.

### Example 14

### Sub-Group I

This sub-group has 54 animals, which are divided in three other lower groups. None of these animals is not burned and is only treated with subcutaneous physiological saline solution as placebo, and physiological saline solutions with different rhamnolipid BAC-3 concentrations two times daily every 12 hours in this way:

1) 18 animals irradiated with LD 50/60 and treated with: a) physiological saline solution (6 animals); b) physiological saline solution with 1.2 mg/kg of rhamnolipid BAC-3; (6 animals) and c) physiological saline solution with 12 mg/kg of rhamnolipid BAC-3; (6 animals).
2) 18 animals irradiated with 2 Gy lower dose of the LD 50/60 dose and treated with: a) physiological saline solution (6 animals); b) physiological saline solution with 1.2 mg/kg of rhamnolipid BAC-3; (6 animals) and c) physiological saline solution with 12 mg/kg of rhamnolipid BAC-3; (6 animals).
3) 18 animals irradiated with 4 Gy lower dose of the LD 50/60 dose and treated with: a) physiological saline solution (6 animals); b) physiological saline solution with 1.2 mg/kg of rhamnolipid BAC-3; (6 animals) and c) physiological saline solution with 12 mg/kg of rhamnolipid BAC-3; (6 animals).

### Example 15

### Sub-Group II

This sub-group consists of 54 animals. Under full anesthesia, two hours after irradiation all animals are burned until achieving full skin burn wound in 3×7 cm diameter. All animals in this sub-group are treated twice daily every 12 hours. The treatment started 24 hours after irradiation and after full skin burn wound in 3×7 cm diameter. The skin burn wounds are simultaneously treated twice daily every 12 hours with 0.1% (w/w) BAC-3 ointment and with the same formulated placebo ointment. In this sub-group the ointment is formulated in a way that BAC-3 has low penetration ability of the skin and burned tissue.

The treatment is performed in this way:

1) 18 animals irradiated with LD 50/60 and treated with: a) physiological saline solution+placebo ointment; (6 animals); b) physiological saline solution with 1.2 mg/kg of rhamnolipid BAC-3+0.1% w/w BAC-3 ointment (6 animals); and c) physiological saline solution with 12 mg/kg of rhamnolipid BAC-3+0.1% w/w BAC-3 ointment (6 animals).
2) 18 animals irradiated with 2 Gy lower dose of the LD 50/60 dose and treated with: a) physiological saline solution (6 animals) placebo ointment; b) physiological saline solution with 1.2 mg/kg of rhamnolipid BAC-3+0.1% w/w BAC-3 ointment (6 animals) and c) physiological saline solution with 12 mg/kg of rhamnolipid BAC-3+0.1% w/w BAC-3 ointment (6 animals).
3) 18 animals irradiated with 4 Gy lower dose of the LD 50/60 dose and treated with: a) physiological saline solution+placebo ointment (6 animals); b) physiological saline solution with 1.2 mg/kg of rhamnolipid BAC-3+0.1% w/w BAC-3 ointment (6 animals); and c) physiological saline solution with 12 mg/kg of rhamnolipid BAC-3+0.1% w/w BAC-3 ointment (6 animals).

### Example 16

### Sub-Group III

This sub-group consists of 54 animals. Under full anesthesia, two hours after irradiation all animals are burned until achieving full skin burn wound in 3×7 cm diameter. All animals in this sub-group are treated twice daily every 12 hours. The skin burn wounds are simultaneously treated twice daily as animals from sub-group II, every 12 hours simultaneously with 1% (w/w) BAC-3 ointment and with the same formulated placebo ointment. In this sub-group the ointment is formulated in a way that BAC-3 has low penetration ability of the skin and burned tissue.

The treatment is performed in the following way:

1) 18 animals irradiated with LD 50/60 and treated with: a) physiological saline solution+placebo ointment; (6 animals); b) physiological saline solution with 1.2 mg/kg of rhamnolipid BAC-3+1% w/w BAC-3 ointment (6 animals); and c) physiological saline solution with 12 mg/kg of rhamnolipid BAC-3+1% w/w BAC-3 ointment (6 animals).
2) 18 animals irradiated with 2 Gy lower dose of the LD 50/60 dose and treated with: a) physiological saline solution (6 animals) placebo ointment; b) physiological saline solution with 1.2 mg/kg of rhamnolipid BAC-3+1% w/w BAC-3 ointment (6 animals) and c) physiological saline solution with 12 mg/kg of rhamnolipid BAC-3+1% w/w BAC-3 ointment (6 animals).
3) 18 animals irradiated with 4 Gy lower dose of the LD 50/60 dose and treated with: a) physiological saline solution+placebo ointment (6 animals); b) physiological saline solution with 1.2 mg/kg of rhamnolipid BAC-3+1% w/w BAC-3 ointment (6 animals); and c) physiological saline solution with 12 mg/kg of rhamnolipid BAC-3+1% w/w BAC-3 ointment (6 animals).

### Example 17

### Sub-Group IV

This sub-group consists of 54 animals. Under full anesthesia, two hours after irradiation all animal's tibias and surrounding tissues are wounded by scalpel. The wound is 1 cm long. All animals are treated as the animals from sub-group III twice daily every 12 hours. At the same time the wound of tibias and surrounded tissue is treated by 1% BAC-3 w/w ointment which has formulation for high penetration possibility of BAC-3 and identical placebo ointment without BAC-3.

1) 18 animals irradiated with LD 50/60 and treated with: a) physiological saline solution+placebo ointment on the wounded tibia and surrounding tissue; (6 animals); b) physiological saline solution with 1.2 mg/kg of rhamnolipid BAC-3+1% w/w BAC-3 ointment on the wounded tibia and surrounding tissue (6 animals); and c) physiological saline solution with 12 mg/kg of rhamnolipid BAC-3+1% w/w BAC-3 ointment on the wounded tibia and surrounding tissue (6 animals).
2) 18 animals irradiated with 2 GY lower dose of LD 50/60 and treated with: a) physiological saline solution+placebo ointment on the wounded tibia and surrounded tissue; (6 animals); b) physiological saline solution with 1.2 mg/kg of rhamnolipid BAC-3+1% w/w BAC-3 ointment on the wounded tibia and surrounding tissue (6 animals); and c) physiological saline solution with 12 mg/kg of rhamnolipid BAC-3+1% w/w BAC-3 ointment on the wounded tibia and surrounding tissue (6 animals).
3) 18 animals irradiated with 4 GY lower dose of LD 50/60 and treated with: a) physiological saline solution+placebo ointment on the wounded tibia and surrounding tissue; (6 animals); b) physiological saline solution with 1.2 mg/kg of rhamnolipid BAC-3+1% w/w BAC-3 ointment on the wounded tibia and surrounding tissue (6 animals); and c) physiological saline solution with 12 mg/kg of rhamnolipid BAC-3+1% w/w BAC-3 ointment on the wounded tibia and surrounding tissue (6 animals).

### Example 18

### Sub-Group V

This sub-group also has 54 animals too. All animals are infected with C. Albicans (1×10⁷ cfu/rat) ten days after subcutaneous treatment with physiological saline solution as placebo, and physiological saline solutions with different rhamnolipid BAC-3 concentrations two times daily every 12 hours in this way:

1) 18 animals irradiated with LD 50/60 and treated with: a) physiological saline solution (6 animals); b) physiological saline solution with 1.2 mg/kg of rhamnolipid BAC-3 (6 animals); and c) physiological saline solution with 12 mg/kg of rhamnolipid BAC-3 (6 animals).
2) 18 animals irradiated with 2 Gy lower dose of the LD 50/60 dose and treated with: a) physiological saline solution (6 animals); b) physiological saline solution with 1.2 mg/kg of rhamnolipid BAC-3 (6 animals) and c) physiological saline solution with 12 mg/kg of rhamnolipid BAC-3 (6 animals).
3) 18 animals irradiated with 4 Gy lower dose of the LD 50/60 dose and treated with: a) physiological saline solution (6 animals); b) physiological saline solution with 1.2 mg/kg of rhamnolipid BAC— (6 animals); and c) physiological saline solution with 12 mg/kg of rhamnolipid BAC-3 (6 animals).

### Per Oral Administration of Rhamnolipid BAC-3

The treatment is identical in doses, time of administration, full skin burning, wounding tibia and surrounding tissues and causing sepsis with C. Albicans as in the subcutaneous treatment, except that rhamnolipid BAC-3 is administrated per oral.

### Example 19

### First Main Group has 58 Animals Used for Determination of the Parameters Necessary Before Performing Main Experiments

The animals are chosen randomly.

1) Ten animals will serve as a control group and will not be treated.
2) Thirty animals are necessary for determination of LD 50/60 parameters according the following doses: a) ten Sprague Dawley female rats in each group underwent irradiation of the whole body doses:|
a) 6.9 Gy
b) 6.1 Gy
c) 7.5 Gy

Previously determined whole body irradiation dose value LD50/30 for Sprague-Dawley female rats was 6.9 Gy

2) Eighteen animals are not exposed to irradiation, burn wounds, wounding of tibia and surrounding tissue by scalpel and are not infected with C. albicans. This group will serve to determine potential side effects of different concentrations of rhamnolipid BAC-3. In this group animals shall be treated twice daily: every 12 hours with:
a) saline solution (6 animals) as placebo;
b) 1.2 mg/kg of BAC-3 in saline solution;
c) 12 mg/kg of BAC-3 in saline solution.

### Example 20

### The Second Main Group Consists of 270 Female Sprague-Dawley Rats

The animals are chosen randomly and treated twice daily (every 12 hours). After determination of LD 50/60, in the first group of animals are: a) 90 animals irradiated with LD 50/60; b) 90 animals irradiated with 2 Gy lower irradiation value of LD 50/60; c) 90 animals irradiated with 4 Gy lower irradiation value of LD 50/60.

All 270 animals are divided in 5 sub-groups. Animals from each sub-group shall be treated during 90 days. Every animal is treated twice daily every 12 hours with two different concentration of rhamnolipid BAC-3 and with placebo. At the same time burned animals shall be treated with ointment with low penetration possibility. The concentration of BAC-3 in these ointments is (0.1% w/w) or (1% w/w) or placebo ointment. The tibia wounds are treated with ointment with high penetration possibility of rhamnolipid BAC-3 (1% w/w) or (1% w/w) or placebo ointment.

### Example 21

### Sub-Group I

This sub-group consist of 54 animals, which are divided in 3 other lower groups. None of these animals is burned and is only treated with subcutaneous physiological saline solution as placebo, and physiological saline solutions with different rhamnolipid BAC-3 concentrations two times daily every 12 hours in the following way:

1) 18 animals irradiated with LD 50/60 and treated with: a) physiological saline solution (6 animals); b) physiological saline solution with 1.2 mg/kg of rhamnolipid BAC-3; (6 animals) and c) physiological saline solution with 12 mg/kg of rhamnolipid BAC-3; (6 animals).
2) 18 animals irradiated with 2 Gy lower dose of the LD 50/60 dose and treated with: a) physiological saline solution (6 animals); b) physiological saline solution with 1.2 mg/kg of rhamnolipid BAC-3; (6 animals) and c) physiological saline solution with 12 mg/kg of rhamnolipid BAC-3; (6 animals).
3) 18 animals irradiated with 4 Gy lower dose of the LD 50/60 dose and treated with: a) physiological saline solution (6 animals); b) physiological saline solution with 1.2 mg/kg of rhamnolipid BAC-3; (6 animals) and c) physiological saline solution with 12 mg/kg of rhamnolipid BAC-3; (6 animals).

### Example 22

### Sub-Group II

This sub-group consist of 54 animals. Under full anesthesia, two hours after irradiation all animals are burned until achieving full skin burn wound in diameter 3×7 cm. All animals in this sub-group are treated twice daily every 12 hours. The treatment started 24 hours after irradiation and achieved full skin burn wound 3×7 cm in diameter. The skin burn wounds are simultaneously treated twice daily every 12 hours with 0.1% (w/w) BAC-3 ointment and with the same formulated placebo ointment. In this sub-group the ointment is formulated in a way that BAC-3 has low skin and burned tissue penetration ability.

The treatment is performed in the following way:

1) 18 animals irradiated with LD 50/60 and treated with: a) physiological saline solution+placebo ointment; (6 animals); b) physiological saline solution with 1.2 mg/kg of rhamnolipid BAC-3+0.1% w/w BAC-3 ointment (6 animals); and c) physiological saline solution with 12 mg/kg of rhamnolipid BAC-3+0.1% w/w BAC-3 ointment (6 animals).
2) 18 animals irradiated with 2 Gy lower dose of the LD 50/60 dose and treated with: a) physiological saline solution (6 animals) placebo ointment; b) physiological saline solution with 1.2 mg/kg of rhamnolipid BAC-3+0.1% w/w BAC-3 ointment (6 animals) and c) physiological saline solution with 12 mg/kg of rhamnolipid BAC-3+0.1% w/w BAC-3 ointment (6 animals).
3) 18 animals irradiated with 4 Gy lower dose of the LD 50/60 dose and treated with: a) physiological saline solution+placebo ointment (6 animals); b) physiological saline solution with 1.2 mg/kg of rhamnolipid BAC-3+0.1% w/w BAC-3 ointment (6 animals); and c) physiological saline solution with 12 mg/kg of rhamnolipid BAC-3+0.1% w/w BAC-3 ointment (6 animals).

### Example 23

### Sub-Group III

This sub-group consists of 54 animals. Under full anesthesia, two hours after irradiation all animals are burned until achieving full skin burn wound in diameter 3×7 cm. All animals in this sub-group are treated twice daily every 12 hours. The treatment starts 24 hours after irradiation and full skin burn wound in diameter 3×7 cm. The skin burn wounds are simultaneously treated twice daily every 12 hours with 1% (w/w) BAC-3 ointment and with the same formulated placebo ointment. In this sub-group the ointment is formulated in a way that BAC-3 has low skin and burned tissue penetration ability.

The treatment is performed in this way:

1) 18 animals irradiated with LD 50/60 and treated with: a) physiological saline solution+placebo ointment; (6 animals); b) physiological saline solution with 1.2 mg/kg of rhamnolipid BAC-3+1% w/w BAC-3 ointment (6 animals); and c) physiological saline solution with 12 mg/kg of rhamnolipid BAC-3+1% w/w BAC-3 ointment (6 animals).
2) 18 animals irradiated with 2 Gy lower dose of the LD 50/60 dose and treated with: a) physiological saline solution (6 animals) placebo ointment; b) physiological saline solution with 1.2 mg/kg of rhamnolipid BAC-3+1% w/w BAC-3 ointment (6 animals) and c) physiological saline solution with 12 mg/kg of rhamnolipid BAC-3+1% w/w BAC-3 ointment (6 animals).
3) 18 animals irradiated with 4 Gy lower dose of the LD 50/60 dose and treated with: a) physiological saline solution+placebo ointment (6 animals); b) physiological saline solution with 1.2 mg/kg of rhamnolipid BAC-3+1% w/w BAC-3 ointment (6 animals); and c) physiological saline solution with 12 mg/kg of rhamnolipid BAC-3+1% w/w BAC-3 ointment (6 animals).

### Example 24

### Sub-Group IV

This sub-group consists of 54 animals. Under full anesthesia, two hours after irradiation all animal's tibias and surrounding tissues are wounded by scalpel. The wound is 1 cm long. All animals are treated twice daily every 12 hours. At the same time the wound of tibia and surrounding tissue is treated by 1% BAC-3 w/w ointment which has formulation for high penetration possibility of BAC-3 and identical placebo ointment without BAC-3.

1) 18 animals irradiated with LD 50/60 and treated with: a) physiological saline solution+placebo ointment on the wounded tibia and surrounding tissue; (6 animals); b) physiological saline solution with 1.2 mg/kg of rhamnolipid BAC-3+1% w/w BAC-3 ointment on the wounded tibia and surrounding tissue (6 animals); and c) physiological saline solution with 12 mg/kg of rhamnolipid BAC-3+1% w/w BAC-3 ointment on the wounded tibia and surrounding tissue (6 animals).
2) 18 animals irradiated with 2 GY lower dose of LD 50/60 and treated with: a) physiological saline solution+placebo ointment on the wounded tibia and surrounding tissue; (6 animals); b) physiological saline solution with 1.2 mg/kg of rhamnolipid BAC-3+1% w/w BAC-3 ointment on the wounded tibia and surrounding tissue (6 animals); and c) physiological saline solution with 12 mg/kg of rhamnolipid BAC-3+1% w/w BAC-3 ointment on the wounded tibia and surrounding tissue (6 animals).
3) 18 animals irradiated with 4 GY lower dose of LD 50/60 and treated with: a) physiological saline solution+placebo ointment on the wounded tibia and surrounding tissue; (6 animals); b) physiological saline solution with 1.2 mg/kg of rhamnolipid BAC-3+1% w/w BAC-3 ointment on the wounded tibia and surrounding tissue (6 animals); and c) physiological saline solution with 12 mg/kg of rhamnolipid BAC-3+1% w/w BAC-3 ointment on the wounded tibia and surrounding tissue (6 animals).

### Example 25

### Sub-Group V

This sub-group consists 54 of animals too. All animals are infected with C. Albicans (1×10⁷ cfu/rat) ten days after subcutaneous treatment with physiological saline solution as placebo, and physiological saline solutions with different rhamnolipid BAC-3 concentrations two times daily every 12 hours in this way:

1) 18 animals irradiated with LD 50/60 and treated with: a) physiological saline solution (6 animals); b) physiological saline solution with 1.2 mg/kg of rhamnolipid BAC-3 (6 animals); and c) physiological saline solution with 12 mg/kg of rhamnolipid BAC-3 (6 animals).
2) 18 animals irradiated with 2 Gy lower dose of the LD 50/60 dose and treated with: a) physiological saline solution (6 animals); b) physiological saline solution with 1.2 mg/kg of rhamnolipid BAC-3 (6 animals) and c) physiological saline solution with 12 mg/kg of rhamnolipid BAC-3 (6 animals).
3) 18 animals irradiated with 4 Gy lower dose of the LD 50/60 dose and treated with: a) physiological saline solution (6 animals); b) physiological saline solution with 1.2 mg/kg of rhamnolipid BAC-3 (6 animals); and c) physiological saline solution with 12 mg/kg of rhamnolipid BAC-3 (6 animals).

### APPLICABILITY

The above examples of performance show that rhamnolipids (BAC-3) has unique characteristics, which could give the possibility that at the same time successfully cure combination of all, or most of illnesses provoked by explosion of atomic bomb or nuclear devices.

1. The method for the use of rhamnolipid composition consists of one or more rhamnolipids with structure formula 1: 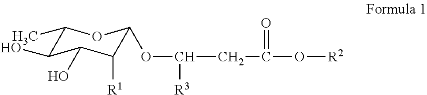
wherein:
R¹═H, unsubstituted α-L-rhamnopyranosyl, α-L-rhamnopyranosyl substituted at the 2 position with
a group of formula —O—C(═O—CH═CH—R⁵, or —O—C(═O)—CH═CH—R⁵;
R²═H, lower alkyl, —CHR⁴—CH₂—COOH or —CHR⁴—CH₂—COOR⁶;
R³=—(CH₂)ₓ—CH₃, wherein x=4-19;
R⁴=—(CH₂)_{y}—CH₃, wherein y=1-19;
R⁵=—(CH₂)_{z}—CH₃, wherein z=1-12; and
R⁶=lower alkyl,
and where said composition is used for the treatment in humans and animals with combination radiation injuries after intentional or non-intentional nuclear catastrophe comprising two or more injuries or illnesses; outstandingly in the combination of radiation illness with burn injuries, mechanical injuries, infection of digestive system, infection of respiratory system neutropenia, sepsis, atherosclerosis, depression, atopic eczema and other illnesses linked with radiation in the way that humans and animals who need such treatment receive effective dose of said composition through adequate way of application.

2. The method for the use of rhamnolipid mixture as claimed in claim 1 where compositions for the treatment of above mentioned combination radiation injuries is the use of the rhamnolipid of the Formula 1 in a way that said compositions comprise: α-L-rhamnopyranosyl-(1,2)-α-L-ramnopyranosyl)-3-hidroksidecanoyl-3-hidroksidecanoic acid and has the following structure Formula 2:

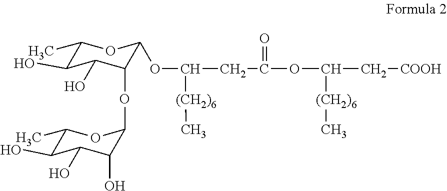

3. The method for the use rhamnolipid composition mixture according claims 1 and 2 wherein said one or more rhamnolipids of Formula 1 are selected from the group consisting of compounds of Formula 1 wherein:
R¹=—O—C(═O)—CH═CH—R⁵, R²=—CHR⁴—CH₂—COOH, R³=—(CH₂)₆—CH₃, R⁴=—(CH₂)₂—CH₃, and R⁵=—(CH₂)₆—CH₃,
or;
R¹=α-L-rhamnopyranosyl substituted at the 2-position by —O—C(═O)—CH═CH—R⁵, R²=—CHR⁴—CH₂—COOH, R³=—(CH₂)₆—CH₃, R⁴=—(CH₂)₆—CH₃, and R⁵=—(CH₂)₆—CH₃;
or;
R¹=—O—C(═O)—CH═CH—R⁵, R²=—CHR⁴—CH₂—COOCH₃, R³=—(CH₂)₆—CH₃, R⁴=—(CH₂)₂—CH₃, and R⁵=—(CH₂)₆—CH₃;
or;
R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

4. The method for the use according to any of the previous claims, in a way that the mixtures of the mentioned rhamnolipids are in the form of clear solution, saline solution, suspension, dispersion, emulsion, cream, paste, powder, infusion solution, oil and ointments, tincture lotion, capsules, pills, suppositories, depo forms, tablets and gel.

5. The method for the use according to claims 1-3, in a way that the applications are intravenous, subcutaneous, per os, intra muscular, or topical on the place of wounds.

6. The method for the use according to claim 5, in a way that the mixture for topical treatment contains one or more rhamnolipids of Formula 1, from previous claims and adequate carrier or any of their combinations which consist of: eucerin, olive oil, sunflower oil and other comestible oils, polyethylene glycol, alcohol, vaseline, water, pharmaceutical alcoholic composition mixtures, physiological solutions, lanoline, milk fat and other fats, propylene glycol, glycerol, stearic acid, vitamins A, D, E and K in combination with oils and similar standard pharmaceutical mixtures used for topical treatment.

7. The method for the use according to claim 4, in a way, that the mixtures contain 0.001%-5% one or more rhamnolipids of Formula 1 related to the whole weight of the composition mixture.

8. The method for the use according to claim 4, in a way that composition mixtures contain 0.01-10% of the one or more rhamnolipids of the Formula 1 related to the whole weight of the composition mixture.

9. The method for the use according to any of the previous claims in a way that the treatment with any of the rhamnolipid compositions must be lasting enough time for successful results of the any illnesses caused by combination radiation injuries in the quantity of 4 mg/kg to 40 mg/kg of the body weight of the treated person or animal 2-4 times/daily, depending of the application way.

10. The method for the use according to any of the previous claims in a way that the treatment with any of the rhamnolipid compositions must be lasting enough time for successful results of any illnesses caused by combination effects of radiation in the quantity of 1 mg/kg to 20 mg/kg of the body weight of the treated person or animal 2-4 times/daily, depending of the application way.

11. The method for the use of rhamnolipid composition mixture which consists of one or more rhamnolipids of the Formula 1: compromising one or more of the structure Formula 1, 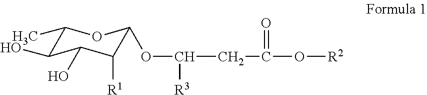
wherein:
R¹═H, unsubstituted α-L-rhamnopyranosyl, α-L-rhamnopyranosyl substituted at the 2 position with
a group of formula —O—C(═O)—CH═CH—R⁵, or —O—C(═O)—CH═CH—R⁵;
R²═H, lower alkyl, —CHR⁴—CH₂—COOH or —CHR⁴—CH₂—COOR⁶;
R³=—(CH₂)ₓ—CH₃, wherein x=4-19;
R⁴=—(CH₂)_{y}—CH₃, wherein y=1-19;
R⁵=—(CH₂)_{z}—CH₃, wherein z=1-12; and
R⁶=lower alkyl,
and where that method with composition mixture is used in humans and/or animals for the treatment of cell and tissue regeneration which comprise: hematopoietic cell and tissue, cover epithelium of skin and mucous membranes, digestive system, respiratory system, hematological system, cardiovascular system, neurological system, muscular-skeletal system after combination radiation injuries which comprise radiation with burns, mechanical injuries, infections of the skin, mucous membranes and organs, atherosclerosis, atopic eczema, and other illnesses which are caused by irradiation; in the way that the treatment of humans and animals who need this treatment receive effective doses of said composition.

12. The method for the use as claimed in claim 11 in the way that for regeneration of the cells and tissues the composition of the mixture is rhamnolipid of the Formula 1 which has formula: (α-L-rhamnopyranosyl-(1,2)-α-L-ramnopyranosyl)-3-hidroksidecanoyl-3-hidroksidecanoic acid) and has the structure Formula 2:

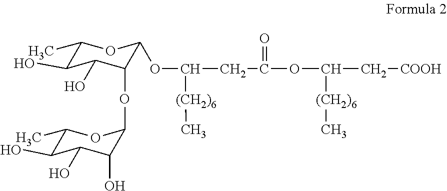

13. The method for the use as claimed in claim 11 in the way that for composition of the used mixture for above mentioned procedure for the regeneration of cell and tissue in the way that are selected from the group consisting of compounds of Formula 1 wherein:
R1=—O—C(═O)—CH═CH—R⁵, R²=—CHR⁴—CH₂—COOH, R³=—(CH₂)₆—CH₃, R⁴=—(CH₂)₂—CH₃, and R⁵=—(CH₂)₆—CH₃, or;
R1=α-L-rhamnopyranosyl substituted at the 2-position by —O—C(═O)—CH═CH—R⁵, R²=—CHR⁴—CH₂—COOH, R³=—(CH₂)₆—CH₃, R⁴=—(CH₂)₆—CH₃, and R⁵=—(CH₂)₆—CH₃, or;
R1=—O—C(═O)—CH═CH—R⁵, R²=—CHR⁴—CH₂—COOCH₃, R³=—(CH₂)₆—CH₃, R⁴=—(CH₂)₂—CH₃, and R⁵=—(CH₂)₆—CH₃, or;
R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.
